(19) United States
(12) Patent Application Publication
(10) Pub. No.: US 2021/0285073 A1
(43) Pub. Date: Sep. 16, 2021

(54) METHOD FOR IDENTIFYING AND TRACING METAL PRODUCTS

(71) Applicant: HYDRO-QUÉBEC, Montréal, Québec (CA)

(72) Inventors: Dominic LEBLANC, Gentilly, Québec (CA); Patrick BOUCHARD, Shawinigan, Québec (CA); Karim ZAGHIB, Longueuil, Québec (CA); Réal LAPORTE, Montreal, Québec (CA)

(73) Assignee: HYDRO-QUÉBEC, Montréal, Québec (CA)

(21) Appl. No.: 17/270,579

(22) PCT Filed: Aug. 23, 2019
(86) PCT No.: PCT/CA2019/051165

§ 371 Date: Feb. 23, 2021

Related U.S. Application Data
(60) Provisional application No. 62/722,514, filed on Aug. 24, 2018.

(51) Int. Cl. C22C 1/02; G01N 33/2028; C22C 23/00; C22C 28/00

(52) U.S. Cl. CPC C22C 1/02; C22C 28/00; C22C 23/00; G01N 33/2028

(57) ABSTRACT

The present invention discloses a method for chemically marking metal products such as metal ingots and other rough or processed metal materials. More specifically, the aim of the present invention is the use of tracers during the method for casting or other methods for manufacturing metal products. These tracers in turn can be identified by physico-chemical methods for confirming the specification of the product, the source of the products or of other similar elements and to assist anti-counterfeiting measures or the identification of production batches or cycles.

### FIELD OF THE INVENTION

This invention has as its purpose a method of chemical marking of metal products such as metal ingots or manufactured alloys. More precisely, this invention pertains to the use of chemical tracers, preferably metal ones, during the casting of metal products. These tracers may be in turn identified by physical-chemical methods in order to confirm the origin of the products and in order to contribute to measures for fighting against counterfeiting or in the identification of the production cycles or batches.

### CONTEXT OF THE INVENTION

Given the high demand for certain metal products in batteries and in electronic materials, such as rare earth elements, lithium metal sheets and other products of the same type, counterfeiting activities are likely to take place. This may have an impact on the quality and the safety of products manufactured from the metal products. Various measures for fighting against counterfeiting have been undertaken to trace back the content, the shipping, the sale, and the origin of the products.

A system of anti-counterfeiting luminescent tags is disclosed in U.S. Pat. No. 7,449,698. Luminescent tags are chemical components in the form of a mainly transparent ink printed or otherwise incorporated into finished products such as luxury items or cigarette packs. The authenticity of the tags is revealed when they are exposed to light energy. The tags then emit a special light spectrum inside at least two strips of luminescence. A device measures the intensities and the luminescence spectrum, compares them to a predetermined standard, and provides an authentic or inauthentic reading.

Another method of identification by tags is disclosed in published American patent application 2015/1022878. It discloses chemical tracer elements incorporated into various resins for the analysis of the source and the batch numbers of raw materials used for the manufacturing of polymer products. The method involves the addition of a series of tracer elements, in specific quantities, to a raw resin. An online analysis tool will shut off the polymer manufacturing equipment if the raw resins are not identified as being authentic.

There exists, in the field of the invention, a need for tags or tracers that may be used in order to identify metal products, given the high temperatures and the challenging conditions of metal foundries and casting operations. In addition, the addition of additives to pure metals leads to certain difficulties that have an impact on the physical and chemical composition of the metal products, which in turn affects their performance and their quality. One of these difficulties is the avoidance of the creation of intermetallic species within a previously pure metal product like an anodic material for batteries, like lithium metal batteries, sodium metal batteries, magnesium metal batteries, and calcium metal batteries. The species could affect the conductivity or other performance characteristics. There is also a need for tags or tracers in which the detectable and/or quantifiable content may be predetermined in order to identify precisely the geographic origin of the production, the production batches, the production cycles, etc.

### SUMMARY OF THE INVENTION

More precisely, this invention provides a marking method of a metal product that allows the precise identification of said method including the steps consisting of: adding one or several omnipresent metal tracer elements of, non-natural origins to a metal product melted in specific concentrations; changing the relative quantities of one or several of the metal tracer elements for each desired production cycle or batch (production batch and/or geographic location of the production site), such that each production cycle or batch has a predetermined combination of metal tracer element ratios.

### Start Here

This invention also provides an identification method of solidified metal products labeled by the use of analytic methods in order to reveal the presence and the concentration of said metallic tracer elements.

Other purposes, benefits, and characteristics of this invention will become more apparent in the reading of the following non-restrictive description of some of its embodiments, given only as examples with references to the FIGURES that accompany them.

### SUMMARY DESCRIPTION OF THE FIGURE

In the appended FIGURE:

FIG. 1 is a schematic diagram of one of the embodiments of this invention.

### DESCRIPTION OF EMBODIMENTS OF THE INVENTION

In one of the embodiments, one or several tracer elements is/are added during the casting process of a chosen metal or alloy (purity in a preferred embodiment: 99% and more, or a chosen metal or alloy). The casting process may be performed in batches or continuously. The high purity metal may be, for example, lithium, magnesium, aluminum, sodium, or calcium or alloys of fixed concentrations of pure metals. The solidified metal ingot obtained by the casting process containing the tracer element(s) may be used, for example, in the manufacturing of metal parts used themselves in the manufacturing of electronic products, including rechargeable batteries such as lithium metal batteries.

The concentration and the presence of a tracer element (or a given exclusive mixture of tracer elements) form a unique signature. In some embodiments, it may be introduced in low concentrations (10 ppmₚ at 1% p) without affecting the conductive or electrochemical properties of the finished products like a lithium metal battery and in adequately high concentrations to make clear identification possible. A chemical analysis or analysis of another nature of the components present in the finished product makes it possible to detect the presence of the profile of the tracer element and to identify whether the finished product, such as a battery, was manufactured with authentic or inauthentic raw materials. The methods of analysis may include, for example, the plasma optical omission spectrometry induced by inductive coupling (ICP-OES) or plasma atomic emission spectrometry induced by inductive coupling (ICP-AES)—including when they are performed after the dissolving of a metal sample in a given lixiviation solvent, the data from X-ray diffraction, mass spectrometry of secondary ions in time-of-flight (ToF-SIMS), fluoroscopy, scanning electron microscopy (MEB), Auger spectroscopy (AES), X-ray photoelectron spectroscopy (XPS), X-ray fluorescence spectroscopy (XRF), chemical reactions with reagents with color reactions, FTIR spectroscopy, etc.

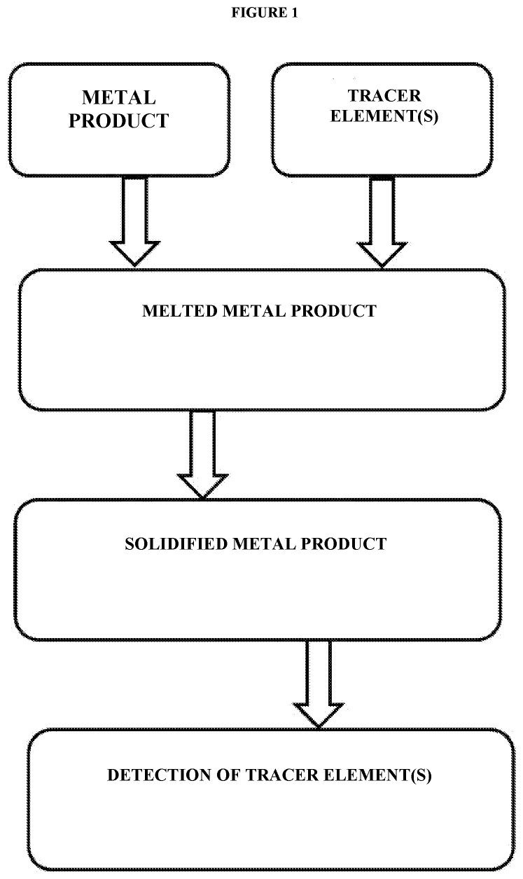

In FIG. 1, a schematic diagram is shown of one embodiment of this invention. As one can see, a melted metal product is enriched by a tracer element or a mixture. Once the metal product has solidified and at some time after production, it is possible to identify with certainty its content, its purity, its production batch, its production site, or any other similar element by chemical detection and analysis and, in some embodiments, the quantification of the tracer element or elements.

The concentration of the tracer element or mixture is chosen for it to be detectable but imperceptible at first sight. Additionally, the tracer element or elements must not generate any secondary intermetallic phase (the concentration must be within the lower range of the limit of solid solubility of the pure metal or alloy) and are chosen from a list of chemical elements that with difficulty may be found “naturally” in commercially pure metals, except where they are already present in very low quantities as impurities or artefacts. In some embodiments, the trace elements are chosen in the family of transition elements (Ti, V, Cr, Mn, Fe, Co, Ni, Cu, Zn, Zr, Nb, Mo, Ru, Rh, Pd, Ag, Cd, Hf, Ta, W, Re, Os, Ir, Pt, Au, Hg, Tl), in the family of alkaline earth metals (Be, Mg, Ca, Sr, Ba) or rare earth elements (Sc, Y, La, Ce, Pr, Nd, Sm, Eu, Gd, Tb, Dy, Ho, Er, Tm, Yb, Lu) or one or several from B, Si, Ge, Sn, Sb, Pb, Bi, In, Ga, Al, Se, Te, Th, Cs, K, Rb, K and Na.

Table 1 presents the basic chemical specification of a Lithium-Magnesium alloy commercial material for lithium alloy batteries.

TABLE 1
Chemical specification of the battery grade lithium/magnesium
alloy (Lectro Max 130 ®, FMC Lithium Li +
Mg 99.9 p % min, Mg 10.0 ± 1.0 p % or 25.0 ± 1.0 p %)

| | Max. concentration |
|---|---|
| Element | (ppmₚ) |
| Ca | 150 |
| Fe | 20 |
| K | 100 |
| N | 300 |
| Na | 100 |
| Si | 100 |

### Example 1

In a stainless steel container, a chemical tracer composed of 110 mg of strontium (99% purity, Sigma Aldrich) and 46.7 g of Lectro Max 130® (Li—Mg10 p %) necessary for the manufacturing of a lithium anode are weighed. The smelting is done at 300° C. in an inert atmosphere. After three hours, the liquid lithium is solidified into a cylinder in the stainless steel mold. The metal lithium cylinder is then extruded in the form of a sheet through a flat die of 500 μm in thickness and 40 mm in width. The extruded sheet of lithium is then cold rolled to an ultra-thin thickness between 20 and 40 μm using a jewelry rolling mill. The ultra-thin lithium metal sheet is dissolved for the purposes of chemical analysis. The chemical analysis is done using an ICP-OES in order to confirm the presence and concentration of the strontium chemical tracer in the batch of lithium anodes produced. The chemical analysis of the tracer element for each casting batch of lithium is then noted. The chemical analysis is presented in Table 2 below. The ultra-thin lithium sheet is used in a 4 cm² Li/SPE/LFP (a lithium iron phosphate cell with solid polymeric electrolyte) electrochemical cell for electrochemical profiling in order to ensure that the electrical performance is not affected by the presence of the chemical tracer.

TABLE 2
ICP-OES chemical analysis in example 1

| | | | |
|---|---|---|---|
| Element | Lithium alloy | Tracer | Li Alloy + Tracer |

| | | | |
|---|---|---|---|
| Li | 88.5% | n/a | 88.3% |
| Mg | 11.1% | 0.0090% | 11.1% |

| Na | 35 | ppmₚ | 55 | ppmₚ | 37 | ppmₚ |
|---|---|---|---|---|---|---|
| K | <1 | ppmₚ | 7 | ppmₚ | <1 | ppmₚ |
| Ca | 45 | ppmₚ | 518 | ppmₚ | 54 | ppmₚ |
| Fe | 44 | ppmₚ | .17 | ppmₚ | 29 | ppmₚ |
| Si | 42 | ppmₚ | n/a | | 48 | ppmₚ |
| Al | 29 | ppmₚ | .11.4 | ppmₚ | 22 | ppmₚ |
| Sr | 2 | ppmₚ | (balance) | | 1983 | ppmₚ |
| Ba | 6 | ppmₚ | 1669 | ppmₚ | 10 | ppmₚ |

The results above show that the Strontium tracer is clearly detected in the finished anode, thereby confirming its identification. No intermetallic species or deterioration of the electrical performance was observed.

### Example 2

In a stainless steel crucible, a chemical tracer composed of 30 mg of ferroniobium (Niobec®) and 180 g of high purity metal magnesium are weighed. The smelting is done at 750° C. in an inert atmosphere. After one hour, the liquid magnesium is solidified into a cylinder in a stainless steel mold. The magnesium ingot is sampled for the purposes of chemical analysis. The sample is dissolved and the chemical analysis is done by ICP-OES in order to confirm the presence and the concentration of the chemical tracer in the batch of magnesium produced. The chemical analysis of the tracer element of each batch of magnesium is noted. The chemical analysis is presented in Table 3 below.

TABLE 2
ICP-OES chemical analysis in example 2

| | Magnesium alloy | | |
|---|---|---|---|
| | Analysis of Mg ingot | Ferroniobium | Mg Alloy + |
| | impurities (ppmₚ) | Tracer | Tracer |
| Element | before adding the tracer | (% p) | (ppmₚ) |
| Al | | 2.0 | 3 |
| Cr | 2 | | 2 |
| Cu | 46 | | 46 |
| Fe | 120 | 26 | 163 |
| Mn | 300 | 1 | 302 |
| Na | 10 | | 10 |
| Nb | <3 | 65.5 | 109 |
| Zn | 56 | | 56 |
| Zr | 10 | | 10 |

The results above show that the Niobium tracer is clearly detected in the finished ingot, thereby confirming the identification of the ingot. No intermetallic species or deterioration of electrical performance was observed.

### Example 3

In a stainless steel container, a chemical tracer composed of 430 mg of zinc (99% purity, Sigma Aldrich) and 45.5 g of Lectro Max 400® metal lithium alloy (battery quality metal lithium, FMC Lithium, Tables 4 and 5) necessary for the manufacturing of a lithium anode are weighed. The smelting is done at 300° C. in an inert argon atmosphere. After three hours, the liquid lithium is solidified in the form of a cylinder in the stainless steel mold. The cylinder of metal lithium is then extruded in the form of a sheet through a flat die of 500 μm in thickness and 40 mm in width. The sheet of extruded lithium is then cold rolled to an ultra-thin thickness between 20 and 40 μm using a jewelry rolling mill. The sheet of ultra-thin metal lithium is dissolved for the purposes of chemical analysis. The chemical analysis is done using an ICP-OES in order to confirm the presence and the concentration of the zinc chemical tracer in the batch of lithium anodes produced. The chemical analysis of the tracer element for each batch of cast lithium is then noted. The chemical analysis is presented in Table 4 below. The sheet of ultra-thin lithium is used in a 4 cm² Li/SPE/LFP electrochemical cell for electrochemical profiling in order to ensure that the electrical performance is not affected by the presence of the chemical tracer.

Table 4 presents the basic chemical specification of metal lithium in a battery grade specification.

TABLE 4
Battery grade metal lithium chemical specification
(Lectro Max 400 ®, FMC Lithium)

| | Max. concentration |
|---|---|
| Element | (ppmₚ) |
| Ca | 150 |
| Cl | 60 |
| Fe | 20 |
| K | 100 |
| N | 300 |
| Na | 100 |
| Si | 100 |

Table 5 presents the chemical analysis of 3 different batches of battery grade metal lithium in order to confirm the natural presence of each element in the commercial battery grade metal lithium.

TABLE 5
Chemical analysis, by ICP-OES, of battery grade metal
lithium (Lectro Max 400 ®, FMC Lithium)

| | Batch 1 | Batch 2 | Batch 3 |
|---|---|---|---|
| Element | (ppmₚ) | (ppmₚ) | (ppmₚ) |
| Ag | 1 | <1 | <1 |
| Al | 1 | 1 | 4 |
| As | 1 | 1 | <1 |
| Au | <1 | <1 | <1 |
| B | <1 | <1 | <1 |
| Ba | 22 | 11 | 7 |
| Be | <1 | <1 | <1 |
| Bi | <1 | <1 | <1 |
| Ca | 16 | 20 | 8 |
| Cd | <1 | <1 | <1 |
| Ce | <1 | <1 | <1 |
| Co | <1 | <1 | <1 |
| Cr | 1 | 2 | 1 |
| Cs | 22 | 11 | 7 |
| Cu | 1 | 2 | 2 |
| Dy | <1 | <1 | <1 |
| Er | <1 | <1 | <1 |
| Eu | <1 | <1 | <1 |
| Fe | 9 | 9 | 9 |
| Ga | <1 | <1 | <1 |
| Gd | <1 | <1 | <1 |
| Ge | <1 | <1 | <1 |
| Hf | <1 | <1 | <1 |
| Hg | <1 | <1 | <1 |
| Ho | <1 | <1 | <1 |
| In | 1 | 1 | <1 |
| Ir | <1 | 1 | 1 |
| K | 1 | 1 | 1 |
| La | <1 | <1 | <1 |
| Lu | <1 | <1 | <1 |
| Mg | 40 | 20 | 9 |
| Mn | <1 | <1 | <1 |
| Mo | <1 | <1 | <1 |
| Na | 2 | 22 | 33 |
| Nb | 1 | 1 | 1 |
| Nd | <1 | <1 | <1 |
| Ni | 1 | 1 | <1 |
| Os | 1 | <1 | <1 |
| P | <1 | <1 | <1 |
| Pb | <1 | <1 | <1 |
| Pd | <1 | <1 | <1 |
| Pr | <1 | <1 | <1 |
| Pt | <1 | <1 | <1 |
| Rb | 6 | 6 | 5 |
| Re | <1 | <1 | <1 |
| Rh | <1 | <1 | <1 |
| Ru | <1 | <1 | <1 |
| S | <1 | <1 | <1 |
| Sb | 1 | <1 | <1 |
| Sc | <1 | <1 | <1 |
| Se | 2 | 1 | <1 |
| Sm | <1 | <1 | <1 |
| Sn | <1 | <1 | <1 |
| Sr | 2 | 2 | 1 |
| Ta | <1 | <1 | <1 |
| Tb | <1 | <1 | <1 |
| Te | 2 | 2 | 1 |
| Th | <1 | <1 | <1 |
| Ti | <1 | <1 | <1 |
| Tl | 1 | 1 | 1 |
| Tm | <1 | <1 | <1 |
| U | 9 | 5 | 7 |
| V | <1 | <1 | <1 |
| W | <1 | <1 | <1 |
| Y | <1 | <1 | <1 |
| Yb | <1 | <1 | <1 |
| Zn | 1 | 1 | 2 |
| Zr | <1 | <1 | <1 |

TABLE 6
Chemical analysis of the final anode produced in Example 3

| | Lithium alloy | |
|---|---|---|
| | LectroMax 400 ® before | |
| | adding the tracer | Li Alloy + Tracer |
| Element | (ppmₚ) | Li Anode (ppmₚ) |
| Al | 1 | 3 |
| Ba | 22 | 24 |
| Ca | 16 | 20 |
| Cu | 1 | 1 |
| Fe | 9 | 13 |
| K | 1 | 1 |
| Mg | 40 | 36 |
| Na | 2 | 2 |
| Si | 2 | 4 |
| Sr | 2 | 2 |
| Zn | 1 | 5310 |

The results above (Table 6) show that the zinc tracer is clearly detected in the finished anode, thereby confirming its identification. No intermetallic species or deterioration of electrical performance of the alloy was observed.

### Example 4

The same procedure as the one from Example 3 was implemented by using potassium as a chemical tracer. 20 mg of potassium (99% purity, Sigma Aldrich) and 50.2 g of Lectro Max 400® metal lithium (battery grade metal lithium, FMC Lithium) were weighed before the melting. The potassium detected by ICP-OES in the final anode was 255 ppmₚ. This result showing that the potassium tracer is clearly detected in the final anode, thereby confirming its identification. No intermetallic species or deterioration of the electrical performance of the alloy was observed.

### Example 5

The same procedure as the one from Example 3 was implemented by using copper as a chemical tracer. 120 mg of copper (99% purity, Sigma Aldrich) and 53.5 g of Lectro Max 400® metal lithium (battery grade metal lithium, FMC Lithium) were weighed before the melting. The copper detected by ICP-OES in the final anode was 2190 ppmₚ. This result showing that the copper tracer is clearly detected in the final anode, thereby confirming its identification. No intermetallic species or deterioration of the electrical performance of the alloy was observed.

In some embodiments, the relative quantities of one or several chemical tracer elements may be set for each desired product specification or production origin such as production batches or production cycles or other data related to the melted metal product, so that each production of solidified metal product has a predetermined and detectable content and, if so desired, a unique and detectable content of tracer elements.

The scope of the claims should not be limited by the embodiments presented in the examples, but should be rather interpreted in the widest sense consistent with the description in general.

1. A chemical marking method of a metal product with information on the product specifications, the production origin, and other technical data, said method including the following steps:
a. adding, to a melted metal product, one or several tracer elements in adequately low concentrations to prevent the formation of intermetallic species and adequately high to allow subsequent detection and identification in a solidified metal product; and
b. setting the relative quantities of one or several of said tracer elements for each product specification or product origin desired or other information of the sort, of the melted metal product, such that each solidified metal product batch or production cycle has a predetermined and detectable tracer content composed of the tracer elements.

2. A method as per claim 1 also including the following steps consisting of:
c. solidifying the metal product; and
d. chemically analyzing the metal product in order to detect and identify the tracer element(s).

3. A method as per claim 1 or 2, in which the solidified metal product is Lithium or a Lithium alloy.

4. A method as per claim 1 or 2, in which the solidified metal product is Magnesium or a Magnesium-based alloy.

5. A method as per claim 1 or 2, in which the solidified metal product is Aluminum or an Aluminum-based alloy.

6. A method as per claim 1 or 2, in which the solidified metal product is Sodium or a Sodium-based alloy.

7. A method as per claim 1 or 2, in which the solidified metal product is Calcium or a Calcium-based alloy.

8. A method as per claim 1 or 2, in which the tracer element is a rare earth element, such as Sc, Y, La, Ce, Pr, Nd, Sm, Eu, Gd, Tb, Dy, Ho, Er, Tm, Yb, Lu or a combination or alloy thereof.

9. A method as per claim 1 or 2, in which the tracer element is a transition element such as Ti, V, Cr, Mn, Fe, Co, Ni, Cu, Zn, Zr, Nb, Mo, Ru, Rh, Pd, Ag, Cd, Hf, Ta, W, Re, Os, Ir, Pt, Au, Hg, Tl or a combination or alloy thereof.

10. A method as per claim 1 or 2, in which the tracer element is an element in the family of alkaline earth metals such as Be, Mg, Ca, Sr, Ba or a combination or alloy thereof.

11. A method as per claim 1 or 2, in which the tracer element is chosen from B, Si, Ge, Sn, Sb, Pb, Bi, In, Ga, Al, Se, Te, Th, Cs, K, Rb, Na or a combination or alloy thereof.

12. A method as per claim 1 or 2, in which the tracer element is Strontium.

13. A method as per claim 1 or 2, in which the tracer element is Niobium.

14. A method as per claim 1 or 2, in which the tracer element is Potassium.

15. A method as per claim 1 or 2, in which the tracer element is Zinc.

16. A method as per claim 1 or 2, in which the tracer element is Copper.

17. A method as per any of claims 2 through 16, in which step d is performed by plasma optical omission spectrometry induced by inductive coupling.

18. A method as per any of claims 2 through 16, in which step d is performed by one or several methods of analysis from the following: X-ray diffraction, mass spectrometry of secondary ions in time-of-flight (ToF-SIMS), fluoroscopy, scanning electron microscopy (MEB), Auger spectroscopy (AES), X-ray photoelectron spectroscopy (XPS), X-ray fluorescence spectroscopy (XRF), the chemical reactions with reagents with color reactions, FTIR spectroscopy.

19. A method as per claim 2 or 17, in which step d is performed after dissolving a metal sample in a given lixiviation solvent.

20. A method as per any of claims 1 through 19, in which step b is performed in such a way that the predetermined tracer content has a unique predetermined signature for a production batch or cycle.

21. A method as per any of claims 1 through 20 in which the predetermined tracer content indicates the geographical origin of the production site of the solidified metal product.
